(19) United States
(12) Patent Application Publication
(10) Pub. No.: US 2024/0376421 A1
(43) Pub. Date: Nov. 14, 2024

(54) Artificial tissue for transplantation therapy, drug screening, Manufacturing method thereof, and Manufacturing apparatus therefor

(71) Applicant: INDUSTRY-ACADEMIC COOPERATION FOUNDATION, YONSEI UNIVERSITY, Seoul (KR)

(72) Inventors: Hyungsuk LEE, Seoul (KR); Seung-Woo CHO, Seoul (KR); Byungjun KANG, Seoul (KR); Jisoo SHIN, Seoul (KR); Chanryeol RHYOU, Seoul (KR)

(21) Appl. No.: 18/780,488

(22) Filed: Jul. 23, 2024

Related U.S. Application Data
(62) Division of application No. 16/775,800, filed on Jan. 29, 2020.

(30) Foreign Application Priority Data
Jan. 30, 2019 (KR) 10-2019-0012159

(51) Int. Cl. C12M 1/42; B01L 3/00; C12M 1/12; C12M 3/00

(52) U.S. Cl. CPC C12M 35/04; B01L 3/502792; C12M 21/08; C12M 25/06

(57) ABSTRACT

Present disclosure provides an apparatus for manufacturing an artificial tissue, comprising:
a hydrogel accommodating structure which accommodates hydrogel including cells and has a bottom plate, a container and a cover; and a standing wave adding means for adding a standing wave to the hydrogel accommodated in the hydrogel accommodating structure, wherein cells in the hydrogel are aligned by the adding of a standing wave, and the position of the cells in the hydrogel is controlled by adjusting at least one of the attenuation coefficient of the container and the reflection coefficient of the cover.

### TECHNICAL FIELD

The present disclosure relates to an artificial tissue for transplantation therapy and drug screening, a manufacturing method thereof, and a manufacturing apparatus therefor. More specifically, the present disclosure relates to an artificial tissue in which the position of cells is controlled in length units from several micrometers to several hundreds of micrometers just like in an actual biological tissue, a method of manufacturing an artificial tissue using acoustic waves, and a manufacturing apparatus therefor.

### BACKGROUND ART

Conventionally, in the field of tissue engineering, tissues for therapy were manufactured by creating a scaffold, attaching cells to the inner surface thereof and culturing the cells. However, in general, scaffolds have a mechanical rigidity remarkably greater than the mechanical rigidity of body tissues which ranges from several kPa to several tens of kPa (>MPa), and thus are not suitable for actual treatment.

Also, in general, since scaffolds have an isotropic structure, they have their limitations in imitating biological tissues such as muscles where cells are aligned in one direction. It is also possible to manufacture a cell sheet by combining cells in various forms and apply the artificial tissue formed by the combining to treatment. However, in order to detach the cells forming the sheet from the culture medium, a thermo-responsive polymer which is very expensive needs to be used, thereby incurring high costs. Also, since the cell sheet is mechanically very weak, a highly skilled expert would be required.

In addition to the above, a bioprinting technique of a direct-cell-printing method where artificial tissues are manufactured by printing bioink, which is a material comprising cells and hydrogel, has been reported. This technique is known to be suitable for manufacturing soft tissues of various structures by using various materials. However, there are still problems such as clogging in the nozzle, long manufacturing time, limitations in manufacturing resolution, and low cell viability.

According to a cell manipulation technique using acoustic waves, it is possible to control the position of cells with high resolution while being non-invasive and biocompatible. Also, it is possible to manipulate a large number of cells at the same time, and it is easy to manipulate cells in a hydrogel solution before hardening. Accordingly, there have been reports on methods of manufacturing an artificial tissue by aligning cells in a parallel plate form in a cuboid container. However, the conventional tissue manufacturing techniques based on acoustic waves have limitations. First, since the tissues are manufactured in a container made of a hard material such as glass, it is difficult to take out and transplant the manufactured soft tissue.

In addition, the conventional reports only present aligning cells in a parallel plane, and thus there are limitations in controlling the shape of the alignment. This makes it difficult to imitate biological tissues of a complex form.

In actual biological tissues, cells are aligned in length units from several micrometers to several hundreds of micrometers. According to relevant researches, it has been known that such cell arrangements may control the biological functions of cells/tissues. Accordingly, in order to have functions similar to biological tissues, it has been known that the cell alignment in biological tissues needs to be imitated.

### PRIOR ART DOCUMENT

### Patent Document

(Patent document 1) Japanese Patent Laid-Open No. 2012-130920 (Jul. 12, 2012)

### SUMMARY OF DISCLOSURE

### Technical Task

The present disclosure aims to solve the above problems of prior art, and it is an object of the present disclosure to provide an artificial tissue with improved therapeutic effects, a method of manufacturing an artificial tissue using acoustic waves, and a manufacturing apparatus therefor by controlling the cell alignment in a tissue in length units from several micrometers to several hundreds of micrometers and manufacturing a cell alignment similar to an actual biological tissue.

### Means for Solving the Task

In order to achieve the above object, an aspect of the present disclosure provides an apparatus for manufacturing an artificial tissue, comprising: a hydrogel accommodating structure which accommodates hydrogel including cells and has a bottom plate, a container and a cover; and a standing wave adding means for adding a standing wave to the hydrogel accommodated in the hydrogel accommodating structure, wherein cells in the hydrogel are aligned by the adding of a standing wave, and the position of the cells in the hydrogel is controlled by adjusting at least one of the attenuation coefficient of the container and the reflection coefficient of the cover.

According to an embodiment of the present disclosure, the hydrogel accommodating structure may be detachable, and may be disassembleable into the bottom plate, the container and the cover.

According to an embodiment of the present disclosure, the container may be made of a material attachable to the bottom plate by van der Waals force.

According to an embodiment of the present disclosure, the reflection coefficient of the cover may be calculated by

$$R = {( \frac{( {Z}_{2} - {Z}_{1} )}{( {Z}_{2} + {Z}_{1} )} )}^{2}$$

where Z₁ is the acoustic impedance of the hydrogel, and Z₂ is the acoustic impedance of the cover.

According to an embodiment of the present disclosure, a vertical pattern may be formed in case the reflection coefficient of the cover is 0.15 or above.

According to an embodiment of the present disclosure, the standing wave adding means may be a surface acoustic wave generating means or an ultrasound transducer.

According to an embodiment of the present disclosure, the standing wave adding means may comprise a substrate and at least a pair of IDT electrodes formed on the substrate.

According to an embodiment of the present disclosure, the at least one pair of IDT electrodes may be aligned in N directions.

According to an embodiment of the present disclosure, the hydrogel accommodating structure may be installed between the at least one pair of IDT electrodes.

According to an embodiment of the present disclosure, the apparatus may further comprise an acoustic coupling medium between the hydrogel accommodating structure and the substrate which the standing wave adding means comprises.

According to an embodiment of the present disclosure, an artificial tissue may be formed by using the apparatus for manufacturing an artificial tissue according to the present disclosure, wherein the position of the cells in the hydrogel is controlled for transplantation therapy and drug screening.

According to an embodiment of the present disclosure, a method of manufacturing an artificial tissue wherein the position of the cells in the hydrogel is controlled may comprise: (a) preparing a hydrogel accommodating structure comprising a bottom plate, a container and a cover, an acoustic wave device, a hydrogel solution including cells, and an acoustic coupling medium; (b) combining the acoustic wave device, the hydrogel accommodating structure, the acoustic coupling medium and the hydrogel solution including cells while injecting the acoustic coupling medium into a space located at an upper side of the acoustic wave device and a lower side of the hydrogel accommodating structure, and injecting the hydrogel solution including cells into the hydrogel accommodating structure; (c) applying acoustic waves to the hydrogel solution including cells and gelling the hydrogel solution including cells after setting the acoustic wave applying conditions; (d) detaching the hydrogel accommodating structure from the acoustic wave device after completing gelation of the hydrogel solution including cells; (e) disassembling the bottom plate, the cover and the container of the hydrogel accommodating structure; and (f) extracting the manufactured artificial tissue.

According to an embodiment of the present disclosure, applying acoustic waves and gelling the hydrogel may be carried out simultaneously in step (c).

According to an embodiment of the present disclosure, the attenuation coefficient of the container and the reflection coefficient of the cover may be set according to the conditions of aligning cells in the hydrogel in step (a).

According to an embodiment of the present disclosure, the directions of the IDT electrode which the acoustic wave device comprises may be set according to the conditions of aligning cells in the hydrogel in step (a).

### Effect of Disclosure

According to an aspect of the present disclosure, the cell alignment in the artificial tissue according to the present disclosure is similar to the cell alignment in actual biological tissues, and thus it is possible to imitate the biological function of biological tissues.

Also, any solution-based hydrogel may be used, and thus it is possible to manufacture an artificial tissue with excellent performance by using the most optimum hydrogel suitable for each tissue.

Further, it is possible to manufacture an artificial tissue by using hydrogel with very low rigidity, and culture and transplant the artificial tissue by extracting the artificial tissue causing little damage.

In addition, it is possible to manufacture a large number of tissues having the same cell alignment repeatedly in a short period of time.

Also, it is possible to keep the time required for manufacturing the tissue constant regardless of the size of the tissue by using acoustic waves.

Further, it is possible to make cell alignments in various forms by varying the shape of the electrode on the substrate and adjusting how to apply the acoustic wave.

The effects of the present disclosure are not limited to the above-mentioned effects, and it should be understood that the effects of the present disclosure include all effects that could be inferred from the configuration of the disclosure described in the detailed description of the disclosure or the appended claims.

### BRIEF DESCRIPTION OF DRAWINGS

FIG. 1A and FIG. 1B are a perspective view and a cross-sectional view illustrating an apparatus for manufacturing an artificial tissue according to an embodiment of the present disclosure;

FIG. 2 illustrates the various types of electrode patterns on the acoustic wave substrate according to an embodiment of the present disclosure;

FIG. 3 illustrates the acoustic potential according to the acoustic attenuation coefficient of the container according to an embodiment of the present disclosure;

FIG. 4A and FIG. 4B illustrate the acoustic potential according to the reflection coefficient of the cover according to an embodiment of the present disclosure and the cell alignment in the artificial tissue;

FIG. 5 is a flow chart illustrating a method of manufacturing an artificial tissue according to an embodiment of the present disclosure;

FIG. 6 schematically illustrates the operation of the apparatus while manufacturing an artificial tissue according to an embodiment of the present disclosure;

FIG. 7 schematically illustrates a process of dissembling the structure and extracting the artificial tissue after manufacturing an artificial tissue according to an embodiment of the present disclosure;

FIG. 8 illustrates the artificial tissue according to an embodiment of the present disclosure and the cell alignment in the artificial tissue; and

FIG. 9 illustrates the blood vessels created by an artificial tissue transplanted to a rat according to an embodiment according to the present disclosure.

### DETAILED MEANS FOR CARRYING OUT THE DISCLOSURE

Hereinafter, the present disclosure will be explained with reference to the accompanying drawings. The present disclosure, however, may be modified in various different ways, and should not be construed as limited to the embodiments set forth herein. Also, in order to clearly explain the present disclosure, portions that are not related to the present disclosure are omitted, and like reference numerals are used to refer to like elements throughout.

Throughout the specification, it will be understood that when a portion is referred to as being “connected” to another portion, it can be “directly connected to” the other portion, or “indirectly connected to” the other portion having intervening portions present. Also, when a component “includes” an element, unless there is another opposite description thereto, it should be understood that the component does not exclude another element but may further include another element.

Hereinafter, examples of the present disclosure will be explained in more detail with reference to the accompanying drawings.

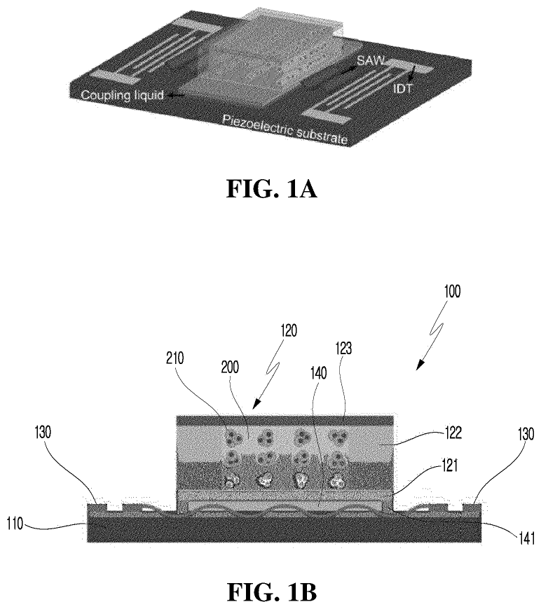

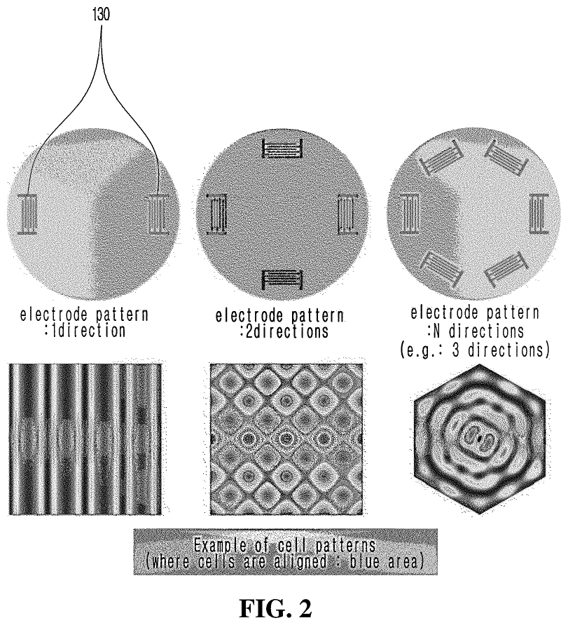

FIG. 1A and FIG. 1B are a perspective view and a cross-sectional view illustrating an apparatus for manufacturing an artificial tissue according to an embodiment of the present disclosure. FIG. 2 illustrates the various types of electrode patterns on the acoustic wave substrate according to an embodiment of the present disclosure

Referring to FIG. 1A and FIG. 1B, the apparatus 100 for manufacturing an artificial tissue comprises: a substrate 110, a hydrogel accommodating structure 120 formed on the substrate 110, and a pair of IDT electrodes 130 symmetrically formed on the substrate 110 having the hydrogel accommodating structure 120 therebetween.

The substrate 110 may be made mainly of piezoelectric material, such as lithium niobite (LiNbO3), quartz or lithium tantalite (LiTaO3), etc., but the material is not particularly limited thereto as far as the material may generate surface acoustic waves (SAW).

A hydrogel accommodating structure 120 for accommodating hydrogel 200 is formed on the substrate 110. Referring to the structure of the hydrogel accommodating structure 120 with reference to FIG. 1A and FIG. 1B, the hydrogel accommodating structure 120 comprises a bottom plate 121 configuring the bottom, a container 122 accommodating hydrogel, and a cover 123 for covering the hydrogel and the container. The hydrogel accommodating structure 120 is detachable from the substrate 110, and is disassembleable into the bottom plate 121, the container 122 and the cover 123. Therefore, as discussed below, the artificial tissue manufactured may be easily extracted from the structure.

Here, preferably, a cover glass may be used as the bottom plate 121, but any material that may serve as a structure for blocking liquid or substances such as hydrogel from the outside may be used.

Preferably, the container 122 may be made of polydimethylsiloxane (hereinafter, “PDMS”), but any material that is attachable to the bottom plate 121 by van der Waals force and may serve as a wall structure creating a space for accommodating liquid or substances such as hydrogel may be used. Accordingly, even when a hydrogel solution is injected to manufacture an artificial tissue, leakage of the solution may be minimized, and the tissue may be easily disassembled after the artificial tissue is manufactured. Also, the shape of the hydrogel is determined by the shape of the container 122, and the container 122 and the cover 123 may lock up the hydrogel, and thus evaporation is minimized.

Referring to FIG. 1A and FIG. 1B again, the apparatus 100 for manufacturing an artificial tissue may further comprise an acoustic coupling medium 140. When the hydrogel accommodating structure 120 is simply placed on the substrate 110, the hydrogel accommodating structure 120 is not in complete contact with the substrate 110, and thus surface acoustic waves cannot be preferably delivered to the hydrogel accommodating structure 120. In order to prevent this, a medium which can mediate the delivery of acoustic waves needs to be arranged between the substrate 110 and the hydrogel accommodating structure 120 to cause acoustic coupling between the structure and the substrate.

Preferably, a fluid such as water (distilled water) or a deformable solid such as PDMS may be used as the acoustic coupling medium 140, but any material may be used as far as the wave energy of the surface acoustic wave delivered through the substrate 110 is delivered. In this case, the strength and pattern of the acoustic wave applied to the structure may vary according to the thickness of the acoustic coupling medium 140. Therefore, the thickness of the medium needs to be optimized through experiments, simulations, etc.

The apparatus 100 for manufacturing an artificial tissue may further comprise a microstructure 141 in order to keep the thickness of the acoustic coupling medium 140 the same every time. The microstructure 141 is interpositioned between the substrate 110 and the bottom plate 121 to serve as a pillar supporting the bottom plate 121. Preferably, the inner space formed by the microstructure 141 may be filled with the acoustic coupling medium 140.

More specifically, after arranging a microstructure 141 of a solid material showing little deformation between the substrate 110 and the hydrogel accommodating structure 120, and placing the hydrogel accommodating structure 120 on the microstructure 141, the acoustic coupling medium 140 may be injected between the hydrogel accommodating structure 120 and the substrate 110. In this case, the thickness of the acoustic coupling medium 140 may be adjusted to the thickness of the microstructure 141. Also, since an acoustic coupling medium 140 is interpositioned between the hydrogel accommodating structure 120 and the substrate 110, the hydrogel accommodating structure 120 may be dissembled from the substrate 110 easily after the process of manufacturing an artificial tissue.

Meanwhile, referring to FIGS. 1 and 2, a standing wave generating means may be formed at both sides of the hydrogel accommodating structure 120 having the hydrogel accommodating structure 120 therebetween. According to an embodiment of the present disclosure, at least one pair of IDT electrodes 130 are adopted as a standing wave generating means, but a stimulating means that may form a pressure field by generating standing waves, for example, an ultrasound transducer may be adopted. Also, the standing wave generating means comprises a substrate 110.

An IDT electrode 130 is formed by depositing conducting material such as gold or aluminum to the substrate 110 made of piezoelectric material. When an alternating current power source is applied to the IDT electrode 130, the IDT electrode 130 transduces the electrical energy to the piezoelectric substrate 110 and then the substrate 110 converted electrical energy to the mechanical waves and eventually generates a surface acoustic waves. The at least one pair of IDT electrodes 130 arranged at both sides of the hydrogel accommodating structure 120 having the hydrogel accommodating structure 120 therebetween generates a surface acoustic wave of the same frequency. The surface acoustic wave of the same frequency overlaps each other, thereby forming a standing wave.

A standing wave is a concept in contrast to a travelling wave, which is a wave proceeding in any direction, meaning a wave having a node of the vibration fixed at a certain position. A standing wave is generated by superposition of waves when waves with the same amplitude and frequency move in opposite directions.

When a standing wave is applied to the hydrogel 200 including cells 210, the cells 210 receive force in the node direction of the standing wave. In this case, an interparticle force is generated between neighboring cells 210. In general, the interparticle force generated between neighboring cells 210 in a solution is an attracting force, and thus the cells 210 cluster at the node. Accordingly, it is possible to manufacture an artificial tissue patterned in a specific alignment having cells 210 in contact with each other. When the cells 210 are in contact with each other, cell-cell junctions may be formed, which are known to promote the proliferation of cells 210 or improve the activity of tissues.

Also, the standing wave is delivered to the hydrogel 200, and inside the hydrogel 200, a standing wave in the longitudinal direction is formed together with a standing wave in the horizontal direction, and through this process, a standing pressure field is generated in the hydrogel 200. In case a standing wave is formed in the hydrogel, the cells 210 are aligned around the node of the standing wave, and thus particles in the hydrogel 200 are aligned at predetermined intervals not only in the horizontal direction but also in the longitudinal (vertical) direction, thereby implementing a 3-dimensional arrangement of the cells 210. In this case, in order to generate the standing pressure field, in addition to standing surface acoustic wave (SSAW), a micro-fabricated ultrasound transducer array, and an acoustic hologram method may be used.

In case a standing wave is formed, the node of the standing wave is formed at an interval of a half-wave length of the surface acoustic wave. Also, since the cells gather around the node of the standing wave, the interval of the cells aligned in the horizontal direction (d_{horizontal}) is determined by the half-wave of the surface acoustic wave.

Also, when the standing wave reaches the hydrogel 200 or fluid, a standing wave in the vertical direction is generated, and particles are aligned at a predetermined internal even in the vertical direction by the standing wave in the vertical direction. The interval of the particles aligned in the vertical direction (d_{vertical}) is the same as the half-wave length of the standing wave in the vertical direction, which is determined by the propagation velocity (V_{liquid}; velocity of sound) of the wave in the hydrogel 200, propagation velocity (V_{SAW}) of the surface acoustic wave in the substrate 110, and the wavelength (λ_{SAW}) of the surface acoustic wave.

Referring to FIG. 2, IDT electrodes 130 may be formed on the substrate 110 in various patterns. More specifically, the IDT electrodes 130 may be aligned on the substrate 110 in various shapes as needed, so that a surface acoustic wave may be applied in various patterns, and a pressure field of various patterns may be formed. In other words, IDT electrodes 130 may be aligned in N directions. In this case, referring to the examples of cell patterns in different directions, the cells may be arranged in the blue area.

In other words, when a surface acoustic wave substrate such as piezoelectric material is used in an acoustic wave generating device in order to form an acoustic field of a desired pattern, electrode patterns may be formed in various directions, and when the wavelength, strength, pulse duration of the acoustic wave are adjusted in each pattern, an acoustic field of a desired pattern may be formed. Accordingly, it is possible to align cells in various forms.

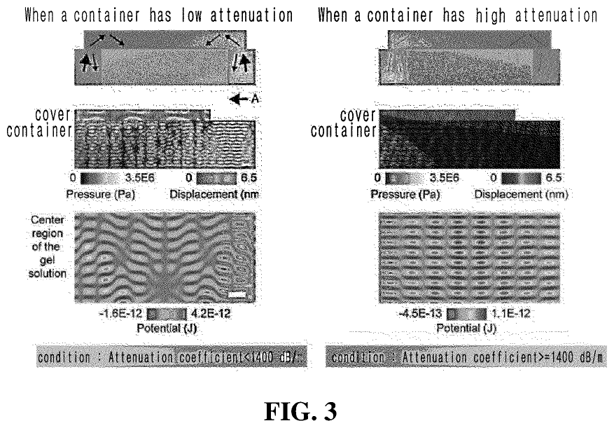

FIG. 3 illustrates the acoustic potential according to the acoustic attenuation coefficient of the container according to an embodiment of the present disclosure.

Referring to FIG. 3, in case of patterning cells 210 in the hydrogel 200, the acoustic properties of the container 122 comprising the hydrogel 200 affect the patterning of cells 210 in the hydrogel 200.

More specifically, since the container 122 is in physical contact with the hydrogel 200, or the bottom plate, 121, it is possible to reflect or transmit acoustic waves at the interface with the hydrogel 200 or the bottom plate 121. Therefore, when attenuation of the acoustic wave in the container 122 is not sufficient, undesired acoustic waves are applied to the inside of the hydrogel 200, thereby distorting the shape of the inner pressure field and aligning cells in undesired patterns. In order to prevent this, the container 122 needs to be manufactured using a substance with sufficient attenuation of acoustic waves. Preferably, rubber, for example PDMS, may be used as a material for the container 122, but any material with sufficient attenuation of acoustic waves may be used.

Referring to FIG. 3 again, preferably, the attenuation coefficient of the container 122 for cell patterning in hydrogel 200 may be 1400 dB/m or above. When the attenuation coefficient is greater than 0 and less than 1400 dB/m, cells are not patterned in the X direction and Z direction. However, when the attenuation coefficient is 1400 dB/m or above, cells are patterned in the X direction and the Z direction. Also, the attenuation coefficient creating a cell patterning may vary according to the thickness of the container, etc.

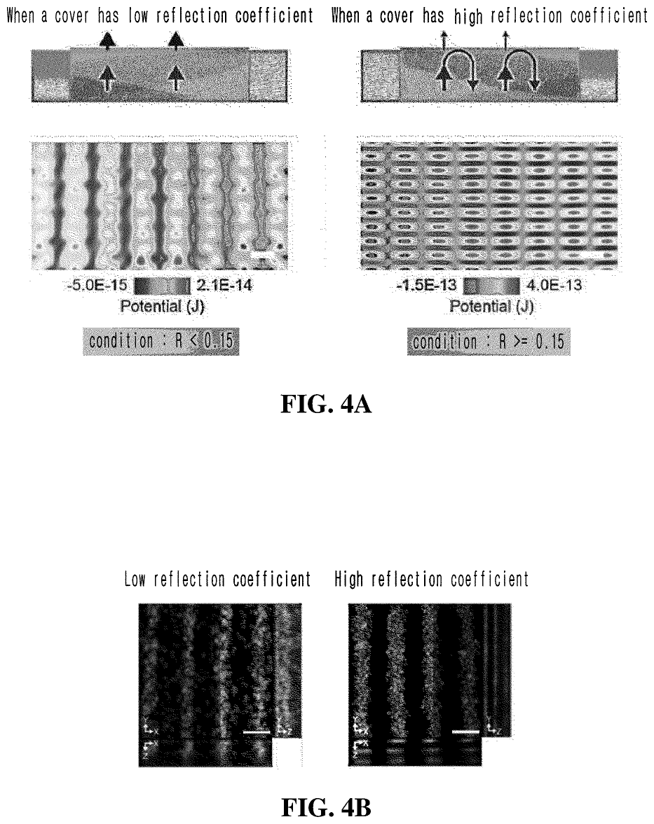

FIG. 4A and FIG. 4B illustrate the acoustic potential according to the reflection coefficient of the cover according to an embodiment of the present disclosure and the cell alignment in the artificial tissue.

Referring to FIG. 4, in case a pressure field is formed in the hydrogel 200, the reflection properties of the cover 123 covering the hydrogel 200 and the container 122 affect the shape of the pressure field. More specifically, when an acoustic field of a desired pattern is formed at an acoustic field generating device beneath the hydrogel 200 and then propagated to the hydrogel 200, the acoustic field is propagated almost at an angle. In this case, it is possible to create pressure fields of different patterns according to the material of the cover 123 covering the hydrogel 200.

The reflection coefficient R of the cover 123 satisfies the following equation where Z₁ is the acoustic impedance of the hydrogel 200, and Z₂ is the acoustic impedance of the cover 123.

$$R = {( \frac{( {Z}_{2} - {Z}_{1} )}{( {Z}_{2} + {Z}_{1} )} )}^{2}$$

Referring to the above equation, when the reflection coefficient R of the cover 123 is greater than 0 and less than 0.15, that is, in order to continuously create the horizontal pattern formed in the acoustic wave generating device in a vertical direction, the upper side of the hydrogel 200 is covered using a cover 123 having an acoustic impedance similar to the hydrogel 200.

When the reflection coefficient R of the cover 123 is 0.15 or above, that is, in order to make patterns in the horizontal direction which are formed in the acoustic wave generating device even in a vertical direction, the upper side of the hydrogel 200 is covered using a cover 123 having acoustic impedance different from the hydrogel 200. In case of patterning in the vertical direction, as stated above, the interval of the particles aligned in the vertical direction (d_{vertical}) is the same as the half-wave length of the standing wave in the vertical direction, which is determined by the propagation velocity (V_{liquid}; velocity of sound) of the wave in the hydrogel 200, propagation velocity (V_{SAW}) of the surface acoustic wave in the substrate 110, and the wavelength (λ_{SAW}) of the surface acoustic wave.

As such, an artificial tissue suitable for the transplant region may be manufactured more efficiently depending on whether there is a vertical pattern in the artificial tissue by controlling the reflection coefficient of the cover 123 as needed and by controlling the interval of the patterns.

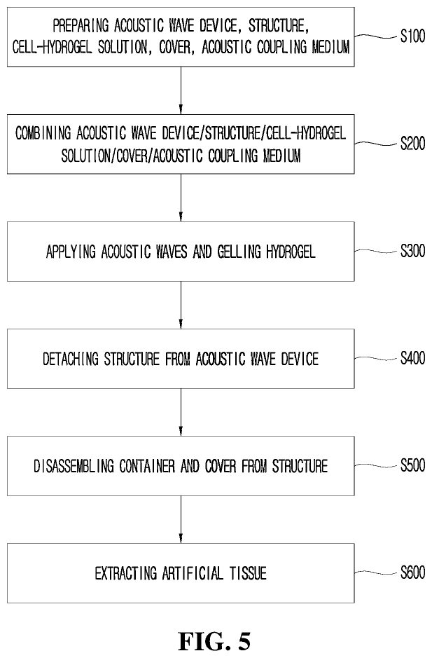

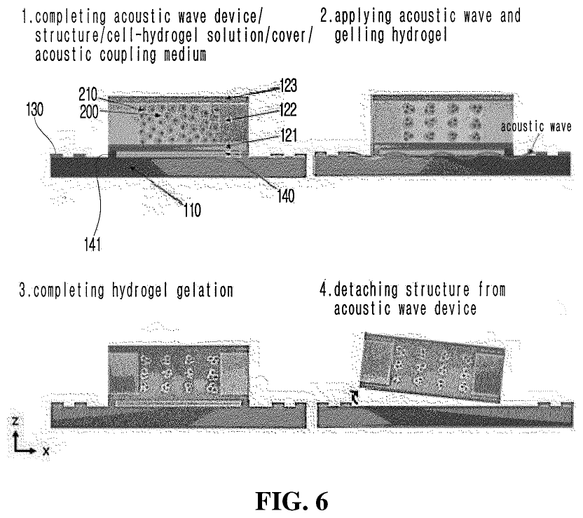

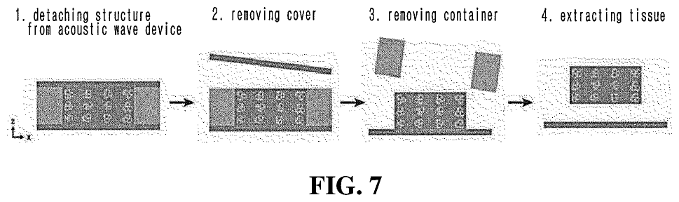

FIG. 5 is a flow chart illustrating a method of manufacturing an artificial tissue according to an embodiment of the present disclosure. FIG. 6 schematically illustrates the operation of the apparatus while manufacturing an artificial tissue according to an embodiment of the present disclosure. FIG. 7 schematically illustrates a process of dissembling the structure and extracting the artificial tissue after manufacturing an artificial tissue according to an embodiment of the present disclosure.

Referring to FIGS. 5 to 7, first, a hydrogel accommodating structure comprising a bottom plate, a container and a cover, an acoustic wave device, a hydrogel solution including cells, and an acoustic coupling medium are prepared (S100). In this case, any hydrogel solution may be used as far as the hydrogel solution is a solution-based hydrogel. Since the cells are aligned by applying a standing pressure field during the gelation of fluid, the hydrogel solution is applicable regardless of the gelation mechanism. Therefore, it is possible to use both the conventionally commercialized hydrogel, and various other hydrogels under development. Accordingly, it is possible to manufacture an artificial tissue of excellent performance using an optimum hydrogel suitable for each tissue. Also, in this case, it is possible to predetermine the attenuation coefficient of the container, the reflection coefficient of the cover, direction of IDT electrode, etc. according to the alignment conditions of the cells in the hydrogel, and prepare the materials accordingly.

Then, the acoustic wave device, the hydrogel accommodating structure, the acoustic coupling medium and the hydrogel solution including cells are combined while injecting the acoustic coupling medium into a space located at an upper side of the acoustic wave device and a lower side of the hydrogel accommodating structure, and injecting the hydrogel solution including cells into the hydrogel accommodating structure (S200). In this case, after a microstructure is formed on the substrate, the structure is arranged on the microstructure, and the space between the structure and the substrate is filled with acoustic coupling medium. Also, after combining the bottom plate and the container of the structure, the hydrogel solution including the cells is injected into the container and the container is covered by a cover. IDT electrodes are arranged on the substrate.

Then, acoustic waves are applied to the hydrogel solution including cells and the hydrogel solution including cells is gelled after setting the acoustic wave applying conditions (S300). In this case, applying acoustic waves and gelling the hydrogel are carried out simultaneously. Therefore, it is possible to control time so that it takes a shorter time to align cells by an acoustic wave than it takes for gelling by controlling acoustic wave applying parameters (e.g.: voltage). Accordingly, in order to manufacture an artificial tissue, it took only the time spent for gelling and the time spent for attaching and detaching the structure. Therefore, it is possible to manufacture a large number of artificial tissues in a short period of time by repeating the method of manufacturing artificial tissues.

Then, the hydrogel accommodating structure is detached from the acoustic wave device after completing gelation of the hydrogel solution including cells (S400), and the bottom plate, the cover and the container of the hydrogel accommodating structure are disassembled (S500). In this case, the order of removing the bottom plate, the cover and the container from the detached hydrogel accommodating structure may vary.

Finally, the artificial tissue manufactured through the above process is extracted (S600).

In case of the conventional technique for manufacturing artificial tissues based on acoustic waves, cells are aligned in a chamber made of a hard material and the tissues are manufactured by hardening hydrogel. Therefore, damage may occur while extracting tissues having low rigidity (<100 kPa). In the present disclosure, by using a structure detachable and disassemblable, the structure can be easily detached from the substrate after the artificial tissue is manufactured, and by disassembling this structure, the hydrogel including cells may be extracted without damage.

Also, in the present disclosure, it takes almost the same time for manufacturing artificial tissues regardless of the size of the tissue. According to the conventional bioprinting technique, it took more time for manufacturing tissues according to the size of the tissue. However, since acoustic waves are propagated at high speed in liquid, even if the size of the solution gets bigger, a standing pressure field can be formed in a very short time. Accordingly, it takes almost the same time for cell alignment by a standing pressure field regardless of the volume of the solution. Therefore, it takes almost the same time for manufacturing artificial tissues regardless of the size of the artificial tissue.

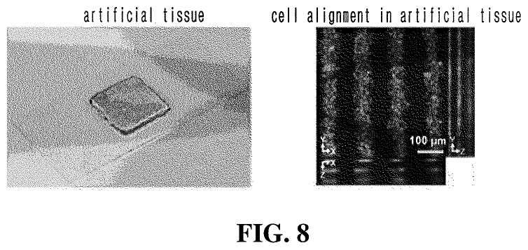

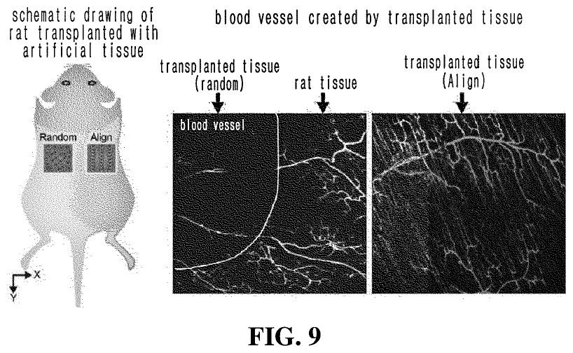

FIG. 8 illustrates the artificial tissue according to an embodiment of the present disclosure and the cell alignment in the artificial tissue. FIG. 9 illustrates the blood vessels created by an artificial tissue transplanted to a rat according to an embodiment according to the present disclosure.

Referring to FIGS. 8 and 9, it may be confirmed that the cells in the artificial tissue manufactured by the apparatus for manufacturing an artificial tissue and the method of manufacturing an artificial tissue of the present disclosure are aligned being patterned in the horizontal and vertical directions, and artificial tissues controlled as above are transplanted to experiment rats.

Referring to FIG. 9, in case of an experiment rat transplanted with an artificial tissue having cells randomly formed in the tissue, revascularization was not properly carried out between the original tissues of the experiment rat and the transplanted artificial tissues. In comparison, in case of an experiment rat transplanted with an artificial tissue having cells formed in the tissue in alignment, it has been confirmed that revascularization was smoothly carried out between the original tissues of the experiment rat and the transplanted artificial tissues.

In other words, when using the artificial tissue manufactured by the apparatus for manufacturing an artificial tissue and a method thereof of the present disclosure, it is possible to manufacture a biological tissue imitating the structure of various tissues such as muscles, blood vessels, nerves, etc. existing in an aligned form in the body and apply the same to treatment.

Also, in case of aligning a drug delivery system in the form of microparticles manufactured by a polymer, a bi-lipid layer, etc. together with cells, it is possible to promote the full growth of artificial tissues, thereby improving and accelerating therapeutic effects.

In addition, in case of aligning a drug delivery system manufactured in the form of microparticles instead of cells, it is possible to develop a structure of a soft material for delivering drugs showing an anisotropic drug release profile.

Further, it is possible to apply the method of manipulating particles in fluid by using acoustic waves regardless of the presence of gravity, and thus it is possible to develop a bioprinter for manufacturing tissues in zero gravity or microgravity.

In addition, in case of manufacturing a muscle tissue using the technique for manufacturing an artificial tissue presented in the present disclosure, it is possible to manufacture a soft actuator with high function, high efficiency, and high energy density which imitates skeletal/cardiac muscles.

The foregoing description of the present disclosure has been presented for illustrative purposes, and it is apparent to a person having ordinary skill in the art that the present disclosure can be easily modified into other detailed forms without changing the technical idea or essential features of the present disclosure. Therefore, it should be understood that the forgoing embodiments are by way of example only, and are not intended to limit the present disclosure. For example, each component which has been described as a unitary part can be implemented as distributed parts. Likewise, each component which has been described as distributed parts can also be implemented as a combined part.

The scope of the present disclosure is presented by the accompanying claims, and it should be understood that all changes or m modifications derived from the definitions and scopes of the claims and their equivalents fall within the scope of the present disclosure.

### DESCRIPTION OF REFERENCE NUMERALS

100 apparatus for manufacturing artificial tissue
110 substrate
120 hydrogel accommodating structure
121 bottom plate
122 container
123 cover
130 IDT electrode
140 acoustic coupling medium
141 microstructure
200 hydrogel
210 cell

What is claimed is:

1. A method of manufacturing an artificial tissue wherein positions of cells in a hydrogel is controlled, the method comprising:
(a) preparing a hydrogel accommodating structure comprising a bottom plate, a container and a cover, an acoustic wave device, a hydrogel solution including cells, and an acoustic coupling medium;
(b) combining the acoustic wave device, the hydrogel accommodating structure, the acoustic coupling medium and the hydrogel solution while injecting the acoustic coupling medium into a space located at an upper side of the acoustic wave device and a lower side of the hydrogel accommodating structure, and injecting the hydrogel solution into the hydrogel accommodating structure;
(c) applying acoustic waves to the hydrogel solution and gelling the hydrogel solution after setting acoustic wave applying conditions;
(d) detaching the hydrogel accommodating structure from the acoustic wave device after completing gelation of the hydrogel solution;
(e) disassembling the bottom plate, the cover and the container of the hydrogel accommodating structure; and
(f) extracting the manufactured artificial tissue.

2. The method of claim 1, wherein the applying of the acoustic waves and the gelling of the hydrogel are carried out simultaneously in step (c).

3. The method of claim 1, wherein an attenuation coefficient of the container and a reflection coefficient of the cover are set according to conditions of aligning cells in the hydrogel in step (a),
wherein the cells in the hydrogel are aligned by adding a standing wave, and the positions of the cells in the hydrogel are controlled 3-dimensionally by adjusting the attenuation coefficient of the container and the reflection coefficient of the cover.

4. The method of claim 3, wherein the acoustic wave device comprises at least one pair of IDT electrodes, and directions of the at least one pair of IDT electrodes are set according to the conditions of the aligning cells in the hydrogel in step (a).

5. The method of claim 1, wherein the reflection coefficient of the cover is calculated by
$$R = {( \frac{( {Z}_{2} - {Z}_{1} )}{( {Z}_{2} + {Z}_{1} )} )}^{2} ,$$
wherein Z₁ is an acoustic impedance of the hydrogel, and Z₂ is an acoustic impedance of the cover.

6. The method of claim 5, wherein a vertical pattern is formed in case the reflection coefficient of the cover is 0.15 or above.
